(19) United States
(12) Patent Application Publication
(10) Pub. No.: US 2009/0305343 A1
(43) Pub. Date: Dec. 10, 2009

(54) Method For Expressing Polypeptides In Eukaryotic Cells Using Alternative Splicing

(75) Inventors: Stéphanie Fallot, Toulouse (FR); Abdelhakim Kharrat, Montgiscard (FR); Philippe Mondon, DonneVille (FR); Khalil Bouayadi, Ramonville Saint-Agne (FR); Patrick Brune, Gif-sur-Yvete (FR); Hervé Prats, Toulouse Cedex (FR); Christian Touriol, Toulouse Cedex (FR)

Correspondence Address: YOUNG & THOMPSON, 209 Madison Street, Suite 500, ALEXANDRIA, VA, 22314, US

(73) Assignees: MILLEGEN, Labege (FR); INSERM (INSTITUT NATIONAL DE LA SANTE ET DE LA RECHERCHE MEDICALE), Paris Cédex 13 (FR)

(21) Appl. No.: 12/301,192

(22) PCT Filed: May 16, 2007
(86) PCT No.: PCT/IB2007/001268

§ 371 Date: Jun. 17, 2009

(30) Foreign Application Priority Data
May 16, 2006 (EP) 06290793.6

(51) Int. Cl. C12P 21/00; C12N 5/10; C12N 1/19; C12N 15/63; A61K 31/711; C07H 21/04

(52) U.S. Cl. 435/69.1; 435/325; 435/348; 435/254.2; 435/320.1; 514/44 R; 536/23.1; 536/23.53

(57) ABSTRACT

This invention relates to an expression cassette for expressing polypeptides in eukaryotic cells using alternative splicing. The expression cassette comprises in 5′ to 3′ downstream direction: a promoter; a sequence transcribed in a 5′ untranslated region (5′UTR); a donor splice site; an intron; a first acceptor splice site; a first cistron encoding a first polypeptide; a second acceptor splice site; a second cistron encoding a second polypeptide; an internal ribosome entry site (IRES) operably linked to a selection marker; and a sequence transcribed in a 3″ untranslated region (3′UTR) including a polyadenylation signal, wherein the polyadenylation signal is unique.

### FIELD OF THE INVENTION

This invention relates to a method for expressing polypeptides in eukaryotic cells using alternative splicing.

### BACKGROUND OF THE INVENTION

Heteromultimeric proteins or polypeptides are composed of different polypeptides. One typical example of these multimeric proteins are the antibodies. They are the result of the association of two heavy chains and two light chains, forming a tetramer complex polypeptide. Other complex proteins are comprised of more than two polypeptides. In the field of multimeric proteins or polypeptides production many approaches have been tested to construct different expression vectors that allow the production of desirable amounts of functional multimeric proteins or polypeptides.

The major difficulty of multimeric polypeptides expression in a transfected cell is the control of the expression ratio between the different monomers which form the multimeric polypeptides. Expression of an unacceptable ratio of antibody light to heavy chain within the same cell may result in a highly inefficient production of the desired multimeric complex or in cell death due to toxicity.

Many research groups around the world have developed several approaches to express multimers in host cells.

In order to answer the need of a vector system where the expression of two coding units could be modulated through a desired ratio of the two polypeptides, WO 2005/089285 describes a vector for the expression of two polypeptides by alternative splicing using one donor splice site and two acceptor splice sites. The splice sites sequences may be mutated in order to modulate the ratio between the two polymers. However, this vector contains a polyadenylation site linked to the first transcription unit. This kind of construct introduces an additional transcription regulation linked to the polyadenylation site. Indeed polyadenylation signal is a specific site for transcription termination (for review see, Proudfoot, 1989) and poly(A) signal strength is directly correlated to termination efficiency (Osheim et al 1999). Another additional expression regulation in the vector described in WO 2005/089285 is the connection between the splicing at the first cistron and the first polyadenylation signal. It has been shown by Niwa et al. 1992 for example, that both splicing and polyadenylation signals are strongly enhanced by each other during transcription termination process. In another work, more than 31 genes were described as expression units in which polyadenylation at promoter-proximal site competes with a splicing reaction to influence expression of multiple mRNAs (cf. Edwalds-Gilbert et al, 1997). These types of regulation using internal poly(A) have also been highlighted in viruses (for review see, Proudfoot, 1996). Hence, the use of an internal poly(A) signal in a vector expression system based on alternative splicing introduces two additional expression regulations i) a bicistronic expression depending on the competition between the internal poly(A) and the adjacent splice acceptor site and ii) an alternative transcription termination introduced by the internal poly(A).

It has also been well known, since 1989, that eukaryotic protein-encoding genes possess poly(A) signals that define the end of the messenger RNA and mediate downstream transcriptional termination by RNA polymerase II (Pol II) (Proudfoot, 1989). 3′ end formation was clearly shown to be linked to transcription both in vitro and in vivo. Although RNA polymerase II is capable of transcribing hundreds of kilobase pairs in a completely processive manner, after transcribing a functional polyadenylation signal the polymerase usually terminates within less than 1 kb (Proudfoot et al., 2002). Moreover, a strong transcriptional pause was found at the precise downstream location to allow efficient cleavage suggesting a coordination of transcription and processing that might block read-through transcription into adjacent genes (Adamson and Price, 2003).

Termination could occur through two mechanisms. The first one in which elongation factors dissociate when the poly(A) signal is encountered, producing termination-competent Pol II, and a second one in which poly(A) site cleavage provides an unprotected RNA 5′ end that is degraded by 5′->3′ exonuclease activities (Xrn2) inducing the dissociation of Pol II from the DNA template. Degradation of the downstream cleavage product by Xrn2 results in transcriptional termination (West et al. 2004).

Differential polyadenylation is a widespread mechanism in higher eukaryotes producing mRNAs with different 3′ ends in different contexts. This involves several alternative polyadenylation sites in the 3′UTR each with different strengths. It is also well known that the efficiency of utilisation of many suboptimal mammalian polyadenylation signals is affected by sequence elements located upstream of the polyadenylation site (AAUAAA), known as upstream efficiency elements (USEs) (Moreira et al., 1995; Hall-Pogar et al. 2005).

According to the transcription termination features linked to the poly(A) signal described above, it appears that WO 2005/089285 describes a system where the first polyadenylation site at the 3′ end of the first cistron plays a major role in the alternative expression of the two polypeptides. This means a high transcription of the first cistron because of the presence of the first poly(A) and a low transcription of a pre-mRNA comprising the two cistrons. Furthermore the work described above do not show any direct evidence by RNA quantification that the expressed polymers result from an alternative RNA splicing between the donor splice site donor and the two acceptor splice sites. It does not show neither any study comparing the RNA amount of the first cistron, the RNA amount of the second cistron and the amount of the non spliced mRNA containing both cistrons. Thus, the internal poly(A) is a major drawback for a system based on alternative splicing to efficiently produce active polypeptide complex.

In WO2005/089285, the vector described harbours two very strong polyadenylation signals (exactly the same sequences) certainly leading mostly to the transcription termination after the first one. If the second site is used, the vector allows the synthesis of the two proteins mainly by an alternative polyadenylation process potentially coupled afterwards with an alternative splicing. Thus, to obtain enough expression of the second polypeptide, the second splicing site of the vector described in WO2005/089285 must be very weak and, by this way, poorly used.

### SUMMARY OF THE INVENTION

The present invention provides an efficient method for expressing polypeptides, especially heteromultimeric polypeptides such as heteroprotein complexes, recombinant antibodies or antibody fragments in host cells using a single expression cassette. The invention provides an expression cassette which may be expressed into an eukaryotic host cell using a single promoter to drive the transcription of a pre-mRNA which can be spliced into two or more mRNAs. In a second step, these mRNAs can be translated into different polypeptides. The expression cassette of the present invention comprises a unique polyadenylation signal located at its 3′ end. Thus any additional regulation involving competition between the splice sites and transcription termination processes are avoided.

### DETAILED DESCRIPTION OF THE INVENTION

The invention provides an expression cassette comprising in 5′ to 3′ downstream direction: a promoter; a sequence transcribed in a 5′ untranslated region (5′UTR); a donor splice site; an intron; a first acceptor splice site; a first cistron encoding a first polypeptide; a second acceptor splice site; a second cistron encoding a second polypeptide; an internal ribosome entry site (IRES) operably linked to a selection marker; and a sequence transcribed in a 3′ untranslated region (3′UTR) including a polyadenylation signal,

wherein the polyadenylation signal is unique, wherein the promoter is operably linked to the first and second cistrons and wherein upon entry into a host cell, said donor splice site splices with said first acceptor splice site, forming a spliced transcript which enables transcription of said first cistron encoding said first polypeptide, and said second acceptor splice site forming a spliced transcript which permits transcription of said second cistron encoding said second polypeptide.

Typically said expression cassette further comprises between said second cistron and said IRES one or more additional acceptor splice sites operably linked to an additional cistron encoding an additional polypeptide wherein upon entry into a host cell, said donor splice site splices with said additional splice acceptor, forming an additional spliced transcript which enables transcription of said additional cistron encoding said additional polypeptide.

The term “expression cassette” refers to a nucleic acid molecule (e.g. DNA, RNA) capable of conferring the expression of a gene product when introduced into a eukaryotic host cell or eukaryotic host cell extract.

The term “promoter” refers to a minimal sequence sufficient to direct transcription. Promoters for use in the invention include, for example, viral, mammalian, insect and yeast promoters that provide for high levels of expression, e.g. the mammalian cytomegalovirus or CMV promoter, the SV40 promoter, or any promoter known in the art suitable for expression in eukaryotic cells.

The term “5′ untranslated region (5′UTR)” refers to an untranslated segment in 5′ terminus of the pre-mRNAS or mature mRNAS. On mature mRNAs, the 5′UTR typically harbours on its 5′ end a 7-methylguanosine cap and is involved in many processes such as splicing, polyadenylation, mRNA export towards the cytoplasm, identification of the 5′ end of the mRNA by the translational machinery and protection of the mRNAs against degradation.

The term “cistron” refers to a segment of nucleic acid sequence that is transcribed and that codes for a polypeptide.

The term “3′ untranslated region (3′UTR)” refers to an untranslated segment in 3′ terminus of the pre-mRNAs or mature mRNAs. On mature mRNAs this region harbours the poly(A) tail and is known to have many roles in mRNA stability, translation initiation, mRNA export . . . .

The term “polyadenylation signal” refers to a nucleic acid sequence present in the mRNA transcripts, that allows for the transcripts, when in the presence of the poly(A) polymerase, to be polyadenylated on the polyadenylation site located 10 to 30 bases downstream the poly(A) signal. Many polyadenylation signals are known in the art and are useful for the present invention. Examples include the human variant growth hormone polyadenylation signal, the SV40 late polyadenylation signal and the bovine growth hormone polyadenylation signal.

The term “splice site” refers to specific nucleic acid sequences that are capable of being recognized by the splicing machinery of a eukaryotic cell as suitable for being cut and/or ligated to a corresponding splice site. Splice sites allow for the excision of introns present in a pre-mRNA transcript. Typically the 5′ portion of the intron is referred to as the donor splice site and the 3′ corresponding splice site is referred to as the acceptor splice site. The term splice site includes, for example, naturally occurring splice sites, engineered splice sites. Engineered splice sites may be mutated sites for example. The mutation of the splice sites enables the control of the ratio between the polypeptides translated from the different populations of transcripts. Splice sites are well known in the art and any may be utilized in the present invention. Consensus sequences for the donor and acceptor splice sites have been defined in the literature.

Typically at least one of said acceptor splice sites comprises any one of the sequences selected from the group consisting of SEQ ID NOS: 1-64 (cf. Table 1).

TABLE 1
Representation of 64 mutated splice site sequences
Mutants sequences for the acceptor splice site

| | |
|---|---|
| SEQ ID N^{o} 1 | CCTTTCTCTCTATAGGT |
| SEQ ID N^{o} 2 | CCTTTCTCTCTAAAGGT |
| SEQ ID N^{o} 3 | CCTTTCTCTCTAGAGGT |
| SEQ ID N^{o} 4 | CCTTTCTCTCTACAGGT |
| Consensus | CCTTTCTCTCCACAGGT |
| SEQ ID N^{o} 5 | |
| SEQ ID N^{o} 6 | CCTTTCTCTCCATAGGT |
| SEQ ID N^{o} 7 | CCTTTCTCTCCAAAGGT |
| SEQ ID N^{o} 5 | CCTTTCTCTCCAGAGGT |
| SEQ ID N^{o} 9 | CCTTTCTCTCGAGAGGT |
| SEQ ID N^{o} 10 | CCTTTCTCTCGACAGGT |
| SEQ ID N^{o} 11 | CCTTTCTCTCGAAAGGT |
| SEQ ID N^{o} 12 | CCTTTCTCTCGATAGGT |
| SEQ ID N^{o} 13 | CCTTTCTCTCAAAAGGT |
| SEQ ID N^{o} 14 | CCTTTCTCTCAACAGGT |
| SEQ ID N^{o} 15 | CCTTTCTCTCAATAGGT |
| SEQ ID N^{o} 16 | CCTTTCTCTCAAGAGGT |
| SEQ ID N^{o} 17 | CCTTCCTCTCTATAGGT |
| SEQ ID N^{o} 18 | CCTTCCTCTCTAAAGGT |
| SEQ ID N^{o} 19 | CCTTCCTCTCTAGAGGT |
| SEQ ID N^{o} 20 | CCTTCCTCTCTACAGGT |
| SEQ ID N^{o} 21 | CCTTCCTCTCCACAGCT |
| SEQ ID N^{o} 22 | CCTTCCTCTCCATAGGT |
| SEQ ID N^{o} 23 | CCTTCCTCTCCAAAGGT |
| SEQ ID N^{o} 24 | CCTTCCTCTCCAGAGGT |
| SEQ ID N^{o} 25 | CCTTCCTCTCGAGAGGT |
| SEQ ID N^{o} 26 | CCTTCCTCTCGACAGGT |
| SEQ ID N^{o} 27 | CCTTCCTCTCGAAAGGT |
| SEQ ID N^{o} 28 | CCTTCCTCTCGATAGGT |
| SEQ ID N^{o} 29 | CCTTCCTCTCAAAAGGT |
| SEQ ID N^{o} 30 | CCTTCCTCTCAACAGCT |
| SEQ ID N^{o} 31 | CCTTCCTCTCAATAGGT |
| SEQ ID N^{o} 32 | CCTTCCTCTCAAGAGGT |
| SEQ ID N^{o} 33 | CCTTACTCTCTATAGGT |
| SEQ ID N^{o} 34 | CCTTACTCTCTAAAGGT |
| SEQ ID N^{o} 35 | CCTTACTCTCTAGAGGT |
| SEQ ID N^{o} 36 | CCTTACTCTCTACAGGT |
| SEQ ID N^{o} 37 | CCTTACTCTCCACAGGT |
| SEQ ID N^{o} 38 | CCTTACTCTCCATAGGT |
| SEQ ID N^{o} 39 | CCTTACTCTCCAAAGGT |
| SEQ ID N^{o} 40 | CCTTACTCTCCAGAGGT |
| SEQ ID N^{o} 41 | CCTTACTCTCGAGAGGT |
| SEQ ID N^{o} 42 | CCTTACTCTCGACAGGT |
| SEQ ID N^{o} 43 | CCTTACTCTCGAAAGGT |
| SEQ ID N^{o} 44 | CCTTACTCTCGATAGGT |
| SEQ ID N^{o} 45 | CCTTACTCTCAAAAGGT |
| SEQ ID N^{o} 46 | CCTTACTCTCAACAGGT |
| SEQ ID N^{o} 47 | CCTTACTCTCAATAGGT |
| SEQ ID N^{o} 48 | CCTTACTCTCAAGAGGT |
| SEQ ID N^{o} 49 | CCTTGCTCTCTATAGGT |
| SEQ ID N^{o} 50 | CCTTGCTCTCTAAAGGT |
| SEQ ID N^{o} 51 | CCTTGCTCTCTAGAGGT |
| SEQ ID N^{o} 52 | CCTTGCTCTCTACAGGT |
| SEQ ID N^{o} 53 | CCTTGCTCTCCACAGGT |
| SEQ ID N^{o} 54 | CCTTGCTCTCCATAGGT |
| SEQ ID N^{o} 55 | CCTTGCTCTCCAAAGGT |
| SEQ ID N^{o} 56 | CCTTGCTCTCCAGAGGT |
| SEQ ID N^{o} 57 | CCTTGCTCTCGAGAGGT |
| SEQ ID N^{o} 58 | CCTTGCTCTCGACAGGT |
| SEQ ID N^{o} 59 | CCTTGCTCTCGAAAGGT |
| SEQ ID N^{o} 60 | CCTTGCTCTCGATAGGT |
| SEQ ID N^{o} 61 | CCTTGCTCTCAAAAGGT |
| SEQ ID N^{o} 62 | CCTTGCTCTCAACAGGT |
| SEQ ID N^{o} 63 | CCTTGCTCTCAATAGGT |
| SEQ ID N^{o} 64 | CCTTGCTCTCAAGAGGT |

The term “cryptic splice site” refers to a site, whose sequence resembles an authentic splice site, and that might be selected instead of an authentic splice site during aberrant splicing. It may be activated if a mutation alters or removes a genuine nearby site. It may be in a coding or non-coding DNA sequence. More particularly, in the vector described in the present invention, any splice site present in the expression cassette, including coding and non-coding sequences, and that is not one of the splice sites described for alternative splicing, i.e. the donor splice site and the acceptor splice site before the first cistron and the acceptor site between the two cistrons, will be referred to as cryptic splice site.

The term “intron” refers to a segment of nucleic acid non-coding sequence that is transcribed and is present in the pre-mRNA but is excised by the splicing machinery based on the sequences of the donor splice site and acceptor splice site, respectively at the 5′ and 3′ ends of the intron, and therefore not present in the mature mRNA transcript. Typically introns have an internal site, called the branch site, located between 20 and 50 nucleotides upstream the 3′ splice site.

The literature on splicing being abundant, it falls within the ability of the skilled person to select, adapt and generate suitable introns and splicing sites in order to construct an expression cassette according to the present invention. Typically splicing sites and introns may be tested for suitability in the present invention by using the methods described in the examples.

The term “internal ribosome entry site (IRES)” refers to a cis-acting sequence able to mediate internal entry of the 40S ribosomal subunit on mRNA upstream of a translation initiation codon (for review, see Hellen and Sarnow, 2001). The presence at the 3′ end of the expression cassette of an IRES operably linked to a selection marker ensures that, in a selected cell, the pre-mRNA is complete and will allow the expression of the different cistrons present in the expression cassette.

The term “operably linked” refers to a juxtaposition wherein the components are in a relationship permitting them to function in their intended manner (e.g. functionally linked).

The term “splice with” refers to the donor splice site interacting with an acceptor splice site to allow splicing of the pre-mRNA by the splicing machinery (e.g. the spliceosome). As described supra, splicing is the excision of a portion of the pre-mRNA (the intron) bounded by a donor splice site and an acceptor splice site. For each transcript, one donor splice site splices with only one acceptor splice site. Alternative splicing means that, within the pool of transcripts the donor splice site may splice with more several different acceptor splice sites. For instance, within the pool of pre-mRNA transcripts, some may be spliced on the first acceptor site and some may be spliced on the second acceptor site. Depending on which acceptor site is used, different mature mRNA transcripts can be generated from a single pre-mRNA transcript, thus generating a heterogeneous pool of transcripts in each transfected cell.

The term “spliced transcript” refers to a mature mRNA transcribed from the expression cassette of the invention which has undergone splicing between the donor splice site and either of the first, second or further acceptor splice sites.

Typically said first, said second and said further polypeptides expressed by said cistrons are all different from each other.

Typically said polypeptides encoded by said cistrons may form a heteromultimeric protein.

In a preferred embodiment the heteromultimeric protein is useful for therapy.

Examples of heteromultimeric proteins include, but are not limited to, heterodimers such as the glycoprotein hormones (e.g. chorionic gonadotropin (CG), thyrotropin (TSH), lutropin (LH), and follitropin (FSH) or members of the integrin family. Heterotetramers consisting of two pairs of identical subunits could also be used. Examples of appropriate heterotetramers include antibodies, the insulin receptor (alpha2 beta2) and the transcription initiation factor TFIIE (alpha2 beta2). By combining different acceptor splice sites, libraries of expression cassettes capable of expressing polypeptides in different ratios can be generated. This allows the efficient expression of many different multimeric proteins.

In a preferred embodiment of the invention, the heteromultimeric protein is an antibody. Antibodies suitable for expressing in a eukaryotic cell using the method of the invention include the five distinct classes of antibody: IgA, IgD, IgG, IgE, and IgM. While all five classes are within the scope of the present invention, the following discussion is generally directed to the class of IgG molecules.

In a preferred embodiment of the invention, said first polypeptide is an antibody light chain or a fragment thereof and said second polypeptide is an antibody heavy chain or a fragment thereof.

In an alternative embodiment of the present invention, said first polypeptide is an antibody heavy chain or a fragment thereof and said second polypeptide is an antibody light chain or a fragment thereof.

An embodiment of the invention relates to a polynucleotide comprising an expression cassette as described previously.

Typically a polynucleotide comprising an expression cassette as described previously is a vector (e.g. a plasmid) which may comprise additional sequences for the propagation of the vector in cells, the entry of the vector into cells and subsequent expression, selectable markers, or any other functional elements. Such elements are well known in the art and can be interchanged as needed using standard molecular biology techniques.

An embodiment of the invention relates to a viral vector comprising the polynucleotide described previously.

The term “viral vector” refers to an attenuated or replication-deficient viral particle. Such viral vectors are useful for inserting the expression cassette of the invention into host cells. Examples of viral vectors are given in WO2005/089285. Adenoviral vector, AAV vector, retroviral vector are examples of commonly used viral vectors.

Typically the skilled person may construct a vector according to the present invention by using an expression cassette as described previously, wherein said cistrons can be easily replaced by other cistrons using different restriction sites located on both sides of said cistrons, and wherein nucleic sequence of said cistrons are cleaned up for putative cryptic splice sites to avoid aberrant splicing events.

An embodiment of the invention relates to an eukaryotic host cell containing a polynucleotide as described previously.

Typically the polynucleotide may be integrated into the chromosomal DNA of said cell.

Examples of suitable eukaryotic host cells are mammalian cells, insect cells and yeast cell.

Typically suitable cells are baby hamster kidney cells, fibroblasts, myeloma cells (e.g., NSO cells), human PER. C6 cells, Chinese hamster ovary cells, COS cells, Spodopterafrugiperda (Sf9) cells, Saccharomyces cells.

An embodiment of the present invention relates to a method of producing polypeptides, the method comprising culturing a cell as described previously in a culture and isolating said polypeptides encoded by said population of transcripts from the culture.

An embodiment of the present invention relates to a polynucleotide or a viral vector as described previously for use in a method for treatment of the human or animal body by therapy wherein said polypeptides encoded by said population of transcripts are therapeutic polypeptides or polypeptides that form a therapeutic heteromultimeric protein.

An embodiment of the present invention relates to the use of a polynucleotide or a viral vector as described previously in the manufacture of a drug for treating a patient in need thereof by gene therapy.

An embodiment of the present invention relates to a method of treating by gene therapy wherein a drug comprising a polynucleotide or a viral vector as described previously is administered to a patient in need thereof.

Typically the drug further comprises a pharmaceutically acceptable carrier.

Gene therapy is a therapy method based on the introduction of a therapeutic gene in the cells of an organism in order to palliate a defective gene involved in a pathology. In a disease where the defective function is the consequence of a defect of heteromultimeric protein or a defect of the products of two or more genes, a polynucleotide according to the invention could be used to treat such a disease. Many vectors such as retroviruses, adenoviruses or plasmids are currently used in gene therapy treatment. Typically, such vectors comprising an expression cassette according to the present invention could be used in a gene therapy protocol. These vectors could be used in a direct in vivo or ex-vivo gene therapy treatment. Examples of diseases which can be treated by gene therapy, of protocol for gene delivery and of treatment regimes and dosages are given in WO2005/089285.

In the following, the invention will be illustrated by means of the following examples as well as the figures.

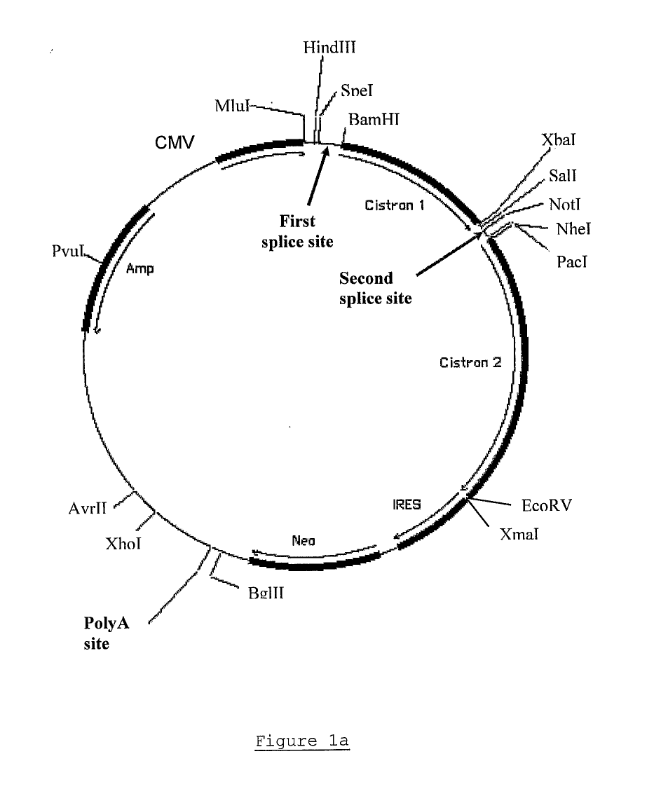

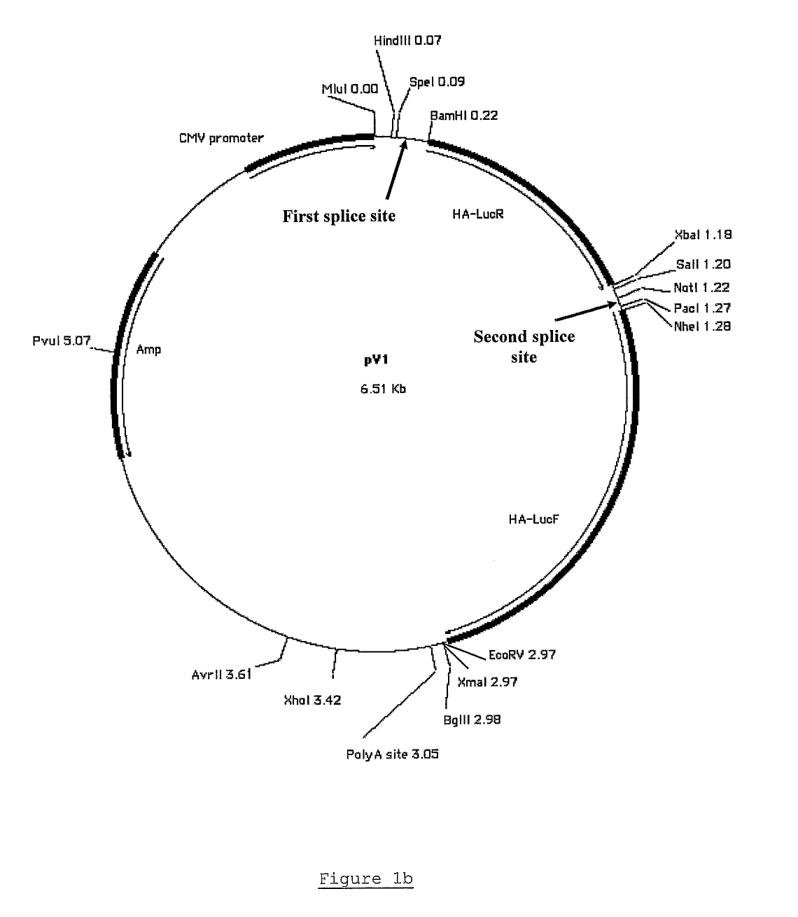

FIG. 1a is a general representation of an example of a vector according to the present invention. The first splice site comprises a donor and an acceptor site. The second splice site comprises a single acceptor site.

FIG. 1b represents a vector containing the HA-tagged reporter genes as the two cistons: HA-LucR (Renillia luciferase) as the first cistron and HA-LucF (Firefly luciferase) as the second cistron.

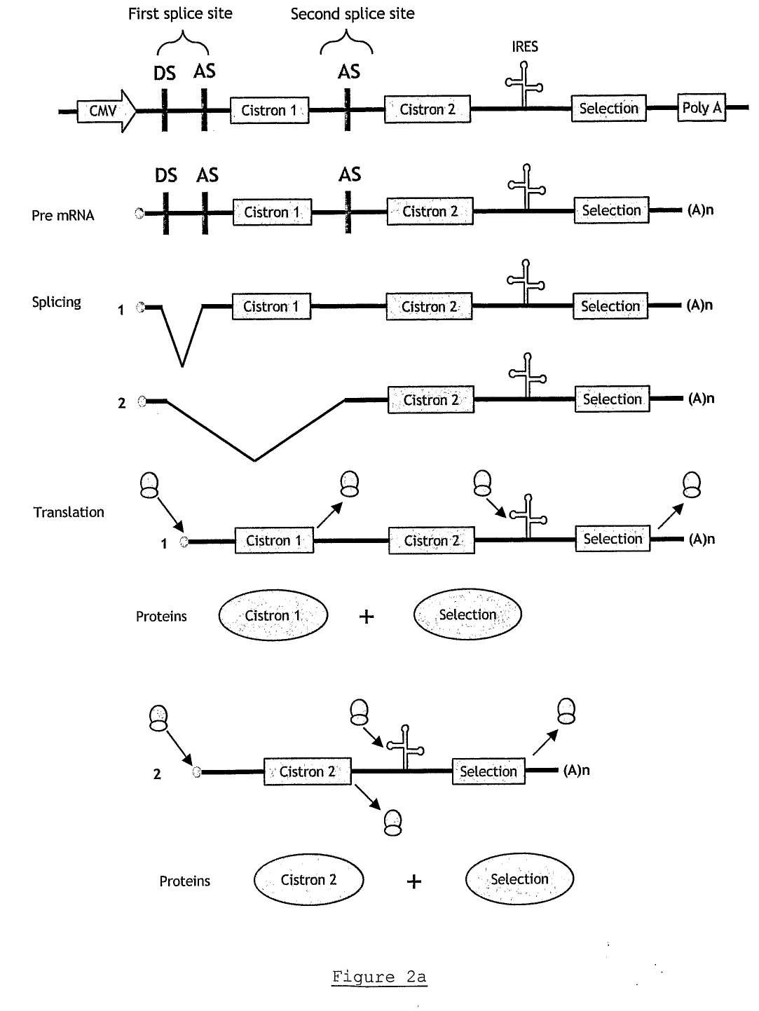

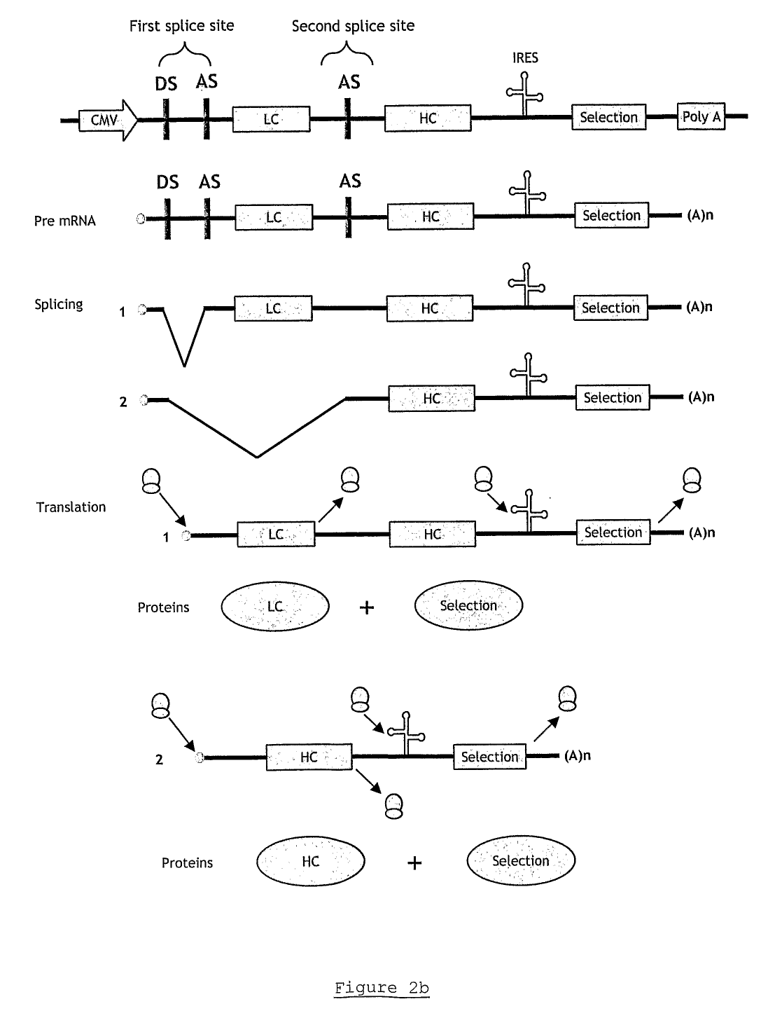

FIG. 2a represents an expression cassette according to the invention and shows a schematic representation of the molecular events leading to the production of the proteins encoded by cistron 1 and cistron 2.

FIG. 2b represents an expression cassette according to a particular embodiment of the invention and shows a schematic representation of the molecular events leading to the production of antibody light chain (LC) and antibody heavy chain (HC).

FIG. 3 represents a schematic representation of the mammalian consensus sequence for an acceptor splice site.

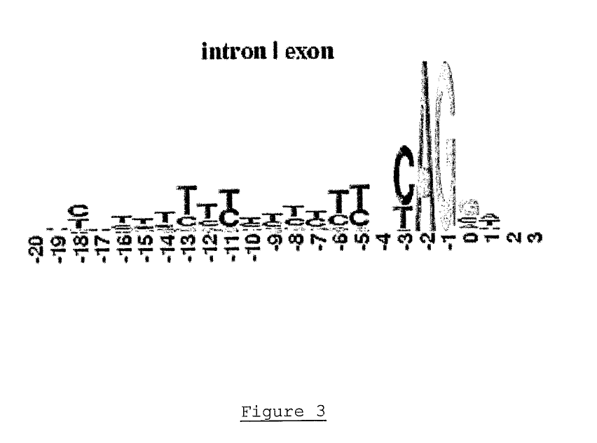

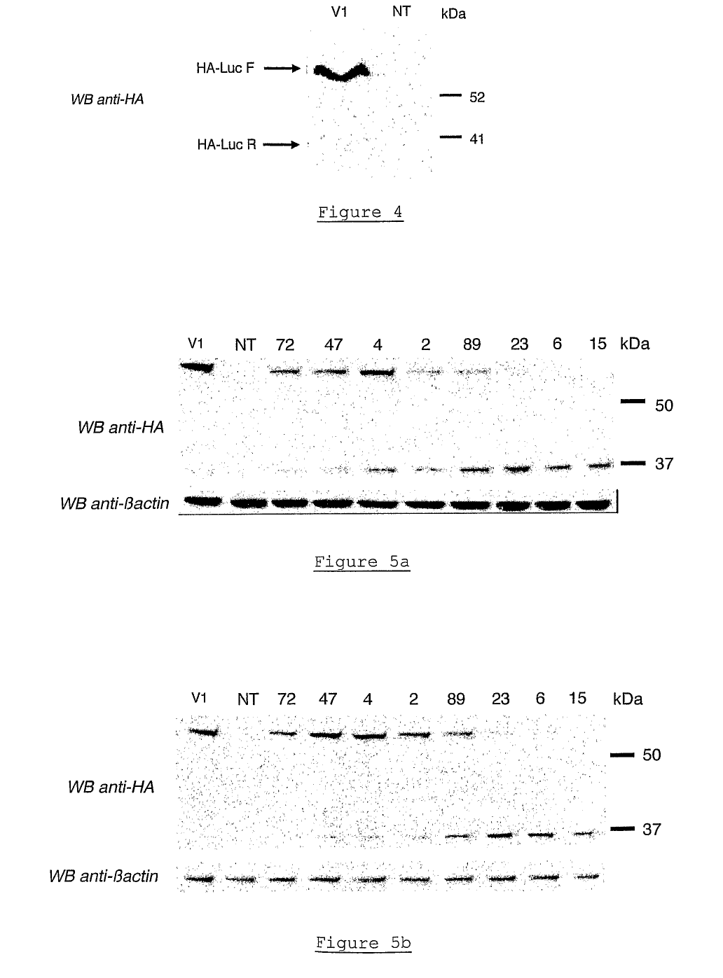

FIG. 4 shows a Western blot analysis on protein extracts from CHO cells transiently transfected with the vector V₁ (pV1). pV₁ is a vector wherein the sequence of the first and second acceptor splice sites are the consensus sequences: CCTTTCTCTCTCACAGGT (SEQ ID No 5).

NT means non transfected cells.

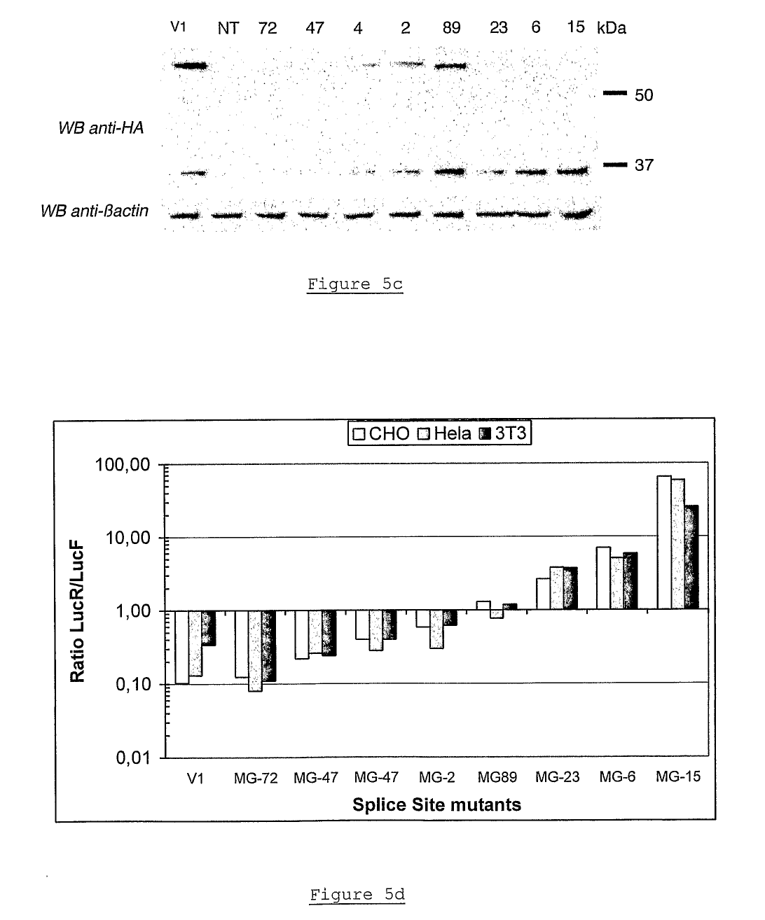

FIG. 5a shows a Western blot analysis on protein extracts from CHO cells transiently transfected with different vectors harbouring mutations in the sequence of the second acceptor splice site. The sequences for the second acceptor splice site of these mutants are listed below (mutated bases are bold):

| | | |
|---|---|---|
| MG-72 (72): | CCTTTCTCTCGACAGGT | (SEQ ID N^{o} 10) |
| MG-47 (47): | CCTTCCTCTCAACAGGT | (SEQ ID N^{o} 30) |
| MG-4 (4): | CCTTCCTCTCGACAGGT | (SEQ ID N^{o} 26) |
| MG-2 (2): | CCTTACTCTCGACAGGT | (SEQ ID N^{o} 42) |
| MG-89 (89): | CCTTGCTCTCAATAGGT | (SEQ ID N^{o} 63) |
| MG-23 (23): | CCTTACTCTCAAAAGGT | (SEQ ID N^{o} 45) |
| MG-6 (6): | CCTTCCTCTCCAGAGGT | (SEQ ID N^{o} 24) |
| MG-15 (15): | CCTTGCTCTCGAGAGGT | (SEQ ID N^{o} 57) |

FIG. 5b shows a Western blot analysis on protein extracts from Hela cells transiently transfected with the same different mutants.

FIG. 5c shows Western blot analysis on protein extracts from NIH-3T3 cells transiently transfected with the same different mutants.

FIG. 5d shows a graphic representation of the LucR/LucF expression ratios obtained with the different mutants in the CHO, HeLa and NIH-3T3 cell lines.

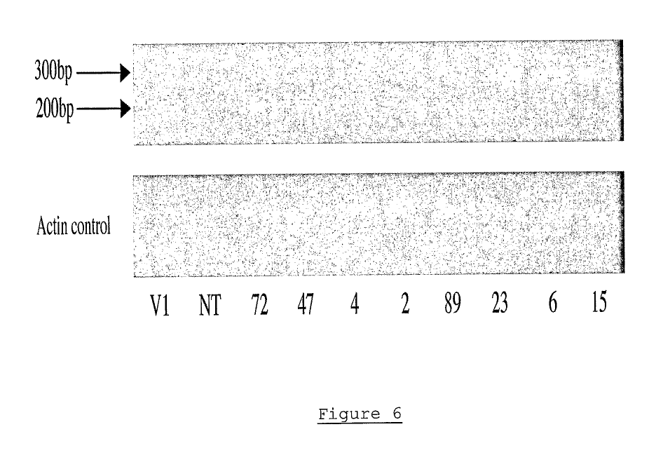

FIG. 6 is a picture of an agarose gel showing the PCR products resulting from RT-PCR experiments on total RNA extracted from CHO cells transfected with the different mutants (30 cycles of PCR).

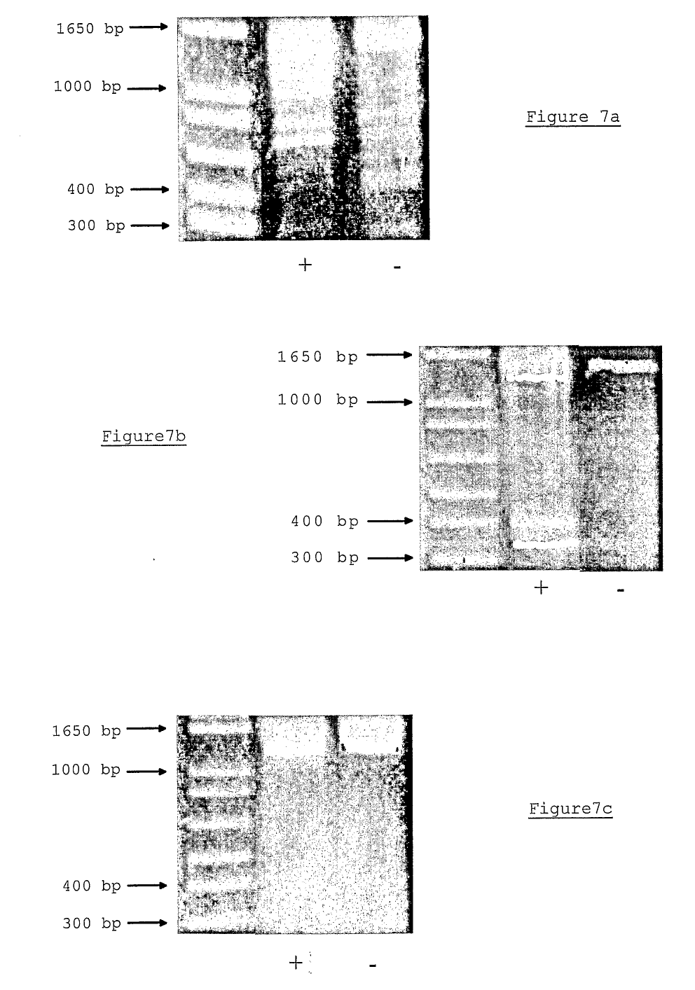

FIG. 7 shows agarose gels showing the PCR products resulting from RT-PCR experiments on total RNA extracted from transfected CHO cells (30 cycles of PCR):

FIG. 7a: transfection with the p1GN-NV vector.

FIG. 7b: transfection with the p1GN-NV mutated on one cryptic splice site.

FIG. 7c: transfection with the p1GN-NV consecutively mutated on several cryptic splice sites.

On each picture, “+” represents the PCR amplification of the cDNAs reverse-transcribed from the total RNA extracted from transfected cells, “−” represents the PCR amplification done in the same conditions on the plasmid used for the transfection (the upper band corresponds to unspliced mRNA).

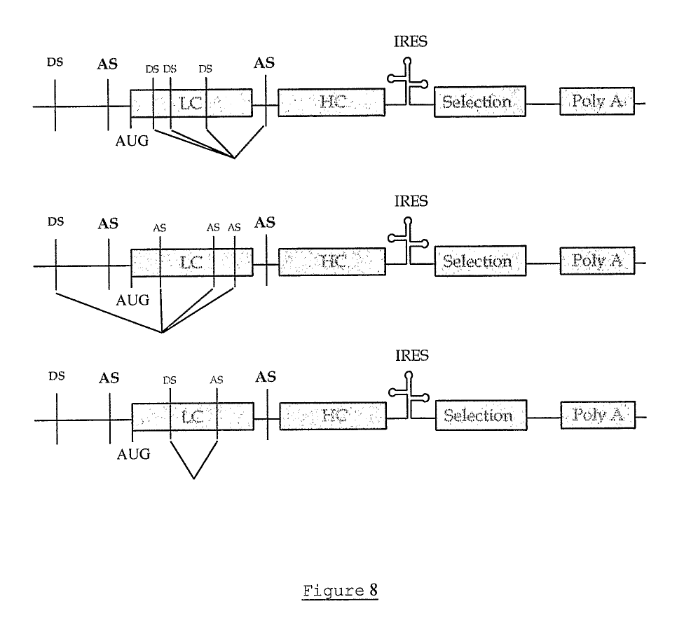

FIG. 8 shows a schematic representation of an example of aberrant splicing events.

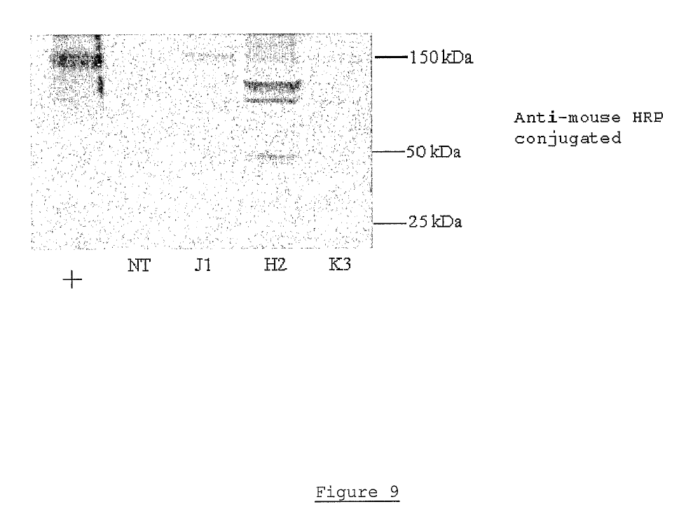

FIG. 9 shows a Western blot analysis on protein extracts from CHO cells transiently transfected with vectors derived from the p1GN-NV and harbouring mutations in the sequence of the first acceptor splice site. K3 corresponds to the p1GN-NV mutated on three different cryptic splice sites and harbouring the consensus sequence for the first acceptor splice site. J1 and H2 are both mutants of K3 and their sequences for the first acceptor splice site are listed below.

J1: CCTTACTCTCGACAGGT (SEQ ID No 42) (mutant MG-2 of example 1)
H2: CCTTGCTCTCGAGAGGT (SEQ ID No 57)(mutant MG-15 of example 1)
NT means non transfected cells.
“+” is a positive control corresponding to the antibody of interest produced by a hybridoma and purified.

### EXAMPLES

In the following description, all molecular biology experiments are performed according to standard protocols (Sambrook J, Fritsch E F and Maniatis T (eds) Molecular cloning, A laboratory Manual 2^{nd} Ed, Cold Spring Harbor Laboratory Press).

### Example 1

### Modulation of Alternative Splicing by Splice Sites Engineering Using Renillia and Firefly Luciferases as Reporter Genes

### Materials and Methods

### 1. Vector Construction:

Basic bicistronic vector construction contains two cistrons which are the two luciferases genes, Renillia luciferase (LucR) and Firefly luciferase (LucF). These reporters genes are both fused to a Hemaglutinin (HA) tag in amino-terminus.

The vector's backbone, obtained from the pCRFL vector (Créancier et al., 2000), includes a CMV promoter, a chimeric intron, a polyadenylation signal and the beta-lactamase gene for selection in prokaryotic cells. The chimeric intron of pCRFL obtained from the pRL-CMV (Promega) comprises the donor splice site from the first intron of the human β-globin gene, and the branch and acceptor splice site from an intron preceding an immunoglobulin gene heavy chain variable region. The sequences of the donor and acceptor splice sites, along with the branch site, have been modified by the manufacturer (Promega) to match the consensus sequences for optimal splicing.

### Intron Sequence:

CAGGTAAGTATCAAGGTTACAAGACAGGTTTAAGGAGACCAATAGAAACT
5′ splice site
GGGCTTGTCGAGACAGAGAAGACTCTTGCGTTTCTGATAGGCACCTATTG
GTCTTACTGACATCCACTTTGCCTTTCTCTCCACAGG
Branch point 3′ splice site

Briefly, pCRFL was digested by XbaI/BglII to remove a sequence containing LucR FGF-2 IRES and LucF genes. After digestion, the backbone portion of the vector described above was gel purified and used for tri-molecular ligation (see below).

LucR was amplified using a polymerase chain reaction (PCR) from the pRL-CMV vector (Promega). The primers used contained restriction sites adjacent to the coding region for further insertion into the backbone plasmid. The 5′ primer also contained the sequence coding for the HA tag in fusion with the luciferase open reading frame. Other restriction sites were also inserted in order to further allow the replacement of the LucR expression cassette by any other protein coding sequence (i.e. BamHI site in 5′ position and NotI site in 3′ position). The 3′ primer contains the sequence of a second acceptor splice site consisting in the following elements: branch point, pyrimidine track and acceptor splice site sequence. This splice site was included between the PacI and NotI restriction sites.

LucR forward primer sequence:

(SEQ ID N^{o} 65)

AAACCTAGGATCCATGTACCCATACGATGTTCCAGATTACGCTN (23)
LucR reverse primer sequence:

(SEQ ID N^{o} 66)

CCTTAATTAACACCTGTGGAGAGAAAGGAAAAGTGGATGTCAGTAAGACC
GCGGCCGCN (21)

where N(23) or N(21) are the nucleotides specific for the LucR gene.

LucF was amplified by PCR using pGL3 vector (Promega) as template. The primers used contained restriction sites adjacent to the coding region for further insertion in the backbone plasmid. The 5′ primer also contained the HA tag coding sequence. Other restriction sites were also inserted in order to further allow the replacement of the LucF expression cassette by any other protein coding sequence and the insertion of the elements IRES/selection gene (i.e. NheI site in 5′ position and XmaI and EcoRV sites in 3′ position).

LucF forward primer sequence:

(SEQ ID N^{o} 67)

CCTTAATTAAGCTAGCATGTACCCATACGATGTTCCAGATTACGCTN
(24)
LucF reverse primer sequence:

(SEQ ID N^{o} 68)

GAAGATCTCCCGGGGATATCN (22)

Both PCR fragments corresponding to the fusion HA-LucR and HA-LucF were gel purified and sequenced.

These two PCR fragments were respectively digested with AvrII/PacI and PacI/BglII and then ligated with the backbone fragment derived from the XbaI/BglII digestion of the pCRFL vector. The resulting vector, named pV3, was checked by sequence analysis.

A second NotI restriction site located 7 bases downstream the LucR stop codon of the constructed pV3 was replaced by the SalI restriction site. The resulting vector, further called pV1, was checked by sequence analysis.

The pV1 vector was then used to transiently transfect CHO cells and evaluate the expression of the two luciferases 24 h after transfection. This analysis was done using classical Western blotting techniques.

### 2. Transient Transfection of CHO, Hela and NIH-3T3 Cells:

1.5 10⁵ CHO cells were plated onto 6 wells dishes 24 h prior transfection. Cells were transfected using Fugene-6 transfection reagent (Roche) according to the manufacturer's instructions (i.e. 8 μl of Fugene reagent for 4 μg of DNA template per well in a serum-free medium).

Hela and NIH-3T3 cells were plated onto 6 wells dishes the day before transfection. Cells were transfected using the JetPEI transfection reagent (Qbiogene) according to the manufacturer's instructions (i.e. 6 μl of JetPEI reagent for 3 μg of DNA template per well in a 150 mM NaCl buffer).

### 3. Western Blot Analysis:

24 h after transfection cells were collected in a phosphate-buffer saline solution and centrifugated. The pellets were resuspended and sonicated in 50 μL of SDS-sample buffer. Protein concentration in the cell lysate was determined using the bicinchoninic acid method (Interchim). Then, samples were boiled at 95° c. for 5 minutes after addition of β-mercaptoethanol and dithiothreitol and 30 μg of total proteins were separated on a NuPAGE 4-12% Bis-Tris gel (Invitrogen). After electrophoretic transfer, the nitrocellulose membranes (Schleicher & Schüell) were blocked with 3% skimmed milk. Luciferases were immunodetected using mouse monoclonal anti-HA (dilution 1:10000) (Babco) as a primary antibody and peroxidase-conjugated sheep anti-mouse (dilution 1:100000) as a secondary antibody (Amersham) and the ECL detection kit (Amersham).

### 4. Design and Methods for Mutating the Second Acceptor Splice Site Sequence:

The following oligonucleotides were used to construct different mutants of the second acceptor splice site:

Forward oligonucleotide:

(SEQ ID N^{o} 69)

GGCCGCGGTCTTACTGACATCCACTTTTCCTTNCTCTTNANAGGTGTAAT
Reverse oligonucleotide:

(SEQ ID N^{o} 70)

TAACACCTNTNAAGAGNAAGGAAAAGTGGATGTCAGTAAGACCGC

These oligonucleotides are complementary and are degenerated on 3 positions of the sequence, i.e. random insertion of one of the four bases occurs during their synthesis. The positions selected for random mutagenesis are:
the first base upstream the intronic 3′ splice site two bases consensus (AG);
the third base upstream the intronic 3′ splice site two bases consensus (AG); and
the ninth base upstream the intronic 3′ splice site two bases consensus (AG).

Considering the consensus sequence of the 3′ splice site shown in FIG. 3, we chose to modify these three bases in order to affect the strength of the 3′ splice site. Indeed changing pyrimidines into purines into the pyrimidine track (e.g. third and ninth bases upstream the two bases AG consensus as we selected) could lead to a slight decrease of the splicing efficiency, while mutating the first base upstream the two bases AG consensus could allow strong modifications in splicing efficiency. Random mutagenesis on these bases leads to 64 sequence possibilities.

Each primer was resuspended to a final concentration of 100 μM in a Tris buffer containing 150 mM NaCl. Equimolar amounts of each oligonucleotide were mixed and hybridization was performed by heating for 10 minutes at 65° C. and cooling at room temperature for 20 minutes. Complete hybridization was checked by running aliquots of each strand as well as the hybrid on a 20% polyacrylamide gel.

Once the complementary oligonucleotides are hybridized, they form a short double stranded DNA fragment with cohesive 5′ and 3′ ends corresponding respectively to the sequence of the NotI and PacI restriction sites.

In a first experiment, the pV1 vector was digested with NotI/XmaI and PacI/XmaI and the corresponding fragments were gel purified and then ligated with the hybridized oligonucleotides in a tri-molecular ligation.

A second possibility used was to digest the pV1 vector with NotI and PacI and then insert the hybridized oligonucleotides during a bi-molecular ligation.

The two experiments were done with different dilutions of the solution of hybridized oligonucleotides from non-diluted (i.e. 100 μM) to 1:100000. The ligation products were used to transform supercompetent TOP10 E. Coli bacteria. Several clones were picked up from LB/agar plates and DNA sequence was determined by sequence analysis in order to identify different mutants for the second acceptor splice site. 53 different mutants were obtained out of the 64 possibilities (cf. Table 1) and were all tested for transient transfection in CHO cells and Western blot analysis to detect expression of the two luciferases.

### 5. RT-PCR Analysis on Transfected CHO Cells:

RT-PCR analyses were performed on transfected CHO total RNA to determine the relative amount of each alternatively spliced luciferase mRNA.

For this experiment, 8×10⁵ cells were seeded on 10 cm culture dishes 24 h prior to transfection. The next day, cells were transfected using Fugene 6 transfection reagent according to the manufacturer's instructions (i.e. 16 μL of Fugene transfection reagent for 8 μg of DNA per dish). 24 h after transfection cells were lysed and total RNA was extracted using the SV Total RNA Isolation system (Promega). Total RNA was quantified by measuring O.D. at 260 nm and samples were then submitted to DNAse treatment (DNA free, Ambion). Similar quantities of total RNA from each sample were then reverse transcribed using the Superscript III First-Strand Synthesis System (Invitrogen) and the resulting cDNA fragments were then amplified by PCR using the following primers:

Primer 1: (Forward primer hybridizing upstream the donor splice site from position 12 to position 31) GAAGTTGGTCGTGAGGCACT (SEQ ID No 71).
Primer 2: (reverse primer hybridizing in the LucR sequence from position 406 to position 426) CATAAATAAGAAGAGGCCGCG (SEQ ID No 72).
Primer 3: (reverse primer hybridizing in the LucF sequence from position 1417 to position 1436) GCAATTGTTCCAGGAACCAG (SEQ ID No 73).

At various PCR cycles (i.e. 18, 20, 22, 24 and 26 cycles) aliquots of PCR products were loaded on a 2% agarose gel. For each sample, a control PCR reaction was performed using human β-actin primers.

### Results:

### A) Alternative Splicing Using Consensus Sequence for Splice Sites

The first transfection experiment was performed with CHO cells using the basic vector (pV1) containing the consensus sequences for the different splice sites. The results are shown in FIG. 4. HA-LucR corresponds to the 37 kDa band and HA-LucF to the 61 kDa band.

It appeared that the two luciferases can be detected in transfected cells and that HA-LucF is quantitatively much more detected than HA-LucR. This result indicates that the second acceptor splice site is more frequently used than the first one by the splicing machinery.

### B) Modulation of Alternative Splicing by Splice Sites Engineering

### a) Western Blot Analysis

In order to regulate the ratio between the two different mRNAs (and consequently regulate the relative expression of the two luciferases) we mutated the sequence of the second acceptor splice site, as described previously, and tested if these modifications had an impact on the choice of the acceptor site selected by the splicing machinery.

As described before, transient transfection was performed on CHO cells with different mutants for the second acceptor splice site. 53 mutants were tested and 8 of them were chosen more particularly. These mutants, when used to transfect cells, generated important variations in the relative expression of the two luciferases (cf. FIG. 5a). The same mutants were also used to transfect other cell types, i.e. Hela cells (human) and NIH-3T3 cells (mouse). Results are shown in table 2 and FIGS. 5b, 5c and 5d.

TABLE 2
LucR/LucF expression ratios induced by
different second acceptor splice sites

| | | Ratio LucR/LucF | | |
|---|---|---|---|---|
| | | | | NIH |
| | | CHO | Hela | 3T3 |
| vectors | Sequence | cells | cells | cells |
| V1 | CCTTTCTCTCCACAGGT | 0.10 | 0.13 | 0.34 |
| (consensus) | | | | |
| MG-72 | CCTTTCTCTCGACAGGT | 0.12 | 0.08 | 0.11 |
| MG-47 | CCTTCCTCTCAACAGGT | 0.22 | 0.26 | 0.24 |
| MG-4 | CCTTCCTCTCGACAGGT | 0.40 | 0.28 | 0.4 |
| MG-2 | CCTTACTCTCGACAGGT | 0.59 | 0.3 | 0.62 |
| MG-89 | CCTTGCTCTCAATAGGT | 1.30 | 0.77 | 1.19 |
| MC-23 | CCTTACTCTCAAAAGGT | 2.65 | 3.78 | 3.71 |
| MG-6 | CCTTCCTCTCCAGAGGT | 7.04 | 5.08 | 5.86 |
| MG-15 | CCTTGCTCTCGAGAGGT | 65.42 | 58.9 | 25.62 |

In all three cell types, different ratios between the HA-LucR and HA-LucF quantities detected can be observed from a large majority of HA-LucF to a large majority of HA-LucR and including intermediate ratios (e.g. ratio close to 1:1, cf. Table 2 and FIG. 5d). This indicates that the expression of the two cistrons can be easily modulated through mutation of the splice sites sequences.

### b) RT-PCR Analysis:

The RT-PCR analysis was performed as previously described after RNA extraction from the CHO cells transfected with the different mutants. The agarose gels corresponding to the PCR products taken after 30 cycles are shown on FIG. 6.

The 200 bp band corresponds to the mRNA transcript resulting from splicing on the second acceptor splice site (HA-LucF when translated). The 300 bp band corresponds to the mRNA transcript resulting from splicing on the first acceptor splice site (HA-LucR when translated).

From mutants MG-72 to MG-15, decreasing amounts of the 200 bp band and increasing amounts of the 300 bp band are observed. These results are in agreement with the pattern of expression of the two proteins shown on Western Blots results (cf. FIGS. 4 and 5). Moreover, it shows that the differential expression of the two proteins is linked to the alternative splicing of the pre-mRNA coding for the two cistrons (HA-LucF and HA-LucR).

### Example 2

### Expression of Antibodies (Light and Heavy Chains as the Two Cistrons) Through Alternative Splicing

### Materials and Methods:

### 1. Vector Construction:

After validation of the vector's functionality with the two reporter genes, the construction was used to express heteromultimeric proteins, more particularly antibodies, as described in a following example. In this case, the two chains of the antibody of interest are expressed from the vector. In the example below, the sequence coding for light chain of the antibody is cloned as the first cistron and the sequence coding for the heavy chain is cloned as the second cistron. This was done using the vector pV1 as a backbone. pV1 was digested by BamHI/XbaI to remove HA-LucR. After digestion, the backbone portion of the vector was gel purified and used for ligation with the light chain sequence (see below). The resulting vector was checked by sequence analysis and then digested by NheI/EcoRV to remove HA-LucF. The corresponding fragment was then gel purified and used for ligation with the heavy chain sequence. The resulting vector was checked by sequence analysis. The sequences of the light and heavy chains were previously amplified by PCR using primers that allow the insertion on both sides of the coding sequences of appropriate restriction sites for further insertion into the plasmid, i.e. BamHI/XbaI for the light chain and NheI/EcoRV for the heavy chain.

In the following example, the antibody expressed from the vector is a monoclonal murine antibody developed in our laboratory. The sequences of the light and heavy chains were then amplified from two plasmids previously constructed in our laboratory containing the cDNA sequences of each chain. The resulting bicistronic vector containing the light and heavy chains of this antibody is further called p1GN-NV. In the same way as the pV1 vector, the p1GN-NV vector was used to transiently transfect CHO cells and evaluate the expression of light chain, heavy chain and entire antibody in the cell lysates and culture supernatant (secreted proteins).

### 2. Transient Transfection of CHO Cells and Analysis of the Expressed Polypeptides:

Each chain of the antibody contains a signal peptide that allows them to be secreted as unassembled chains (light chain only) or whole antibody (heterotetramer). The expression of the antibody's chains is therefore detected in the cell lysates to study the non-secreted proteins and in the cell culture supernatants to study the secreted proteins.

The protocol for the transient transfection of CHO cells and for the detection of the proteins in the cell lysates by Western Blot analysis is the same as described above for the luciferases. Cell extracts may be reduced (addition of β-mercaptoethanol and dithiothreitol before heating) before migration on the NuPAGE gel in order to dissociate the different multimers that may have formed. Migration of the non-reduced samples was also done in order to detect the putative presence of whole antibody molecules (two light chains assembled with two heavy chains) and eventually, many heteromultimeric intermediate species or unassembled free chains. Immunodetection of the different protein complexes on the nitrocellulose membranes is done using a peroxidase-conjugated sheep anti-mouse antibody (dilution 1:100000) (Amersham), or a peroxidase-conjugated goat anti-mouse kappa light chain antibody (dilution 1:10000) (Bethyl Laboratories) and the ECL detection kit (Amersham).

Detection of the secreted polypeptides in the cell culture supernatants was done using several approaches:
precipitation of the whole proteins from culture supernatants using acetone. After transfection, cells were grown in the appropriate medium containing a low percentage of serum (0.2%). 24 h after transfection, cell culture supernatant is collected, centrifugated to remove cells and debris, mixed with 7 volumes of acetone and placed at 20° C. for 3 hours at least. The precipitated proteins are then centrifugated, pellets are dried to remove all traces of acetone and finally the proteins are resuspended with the appropriate volume of SDS-sample buffer.
purification of the whole antibody molecules or antibody fragments directly from culture supernatants using protein A or protein G based purification systems according to the manufacturer's instructions: protein-A Sepharose 4B, Kappalock Sepharose 4B (Zymed, Invitrogen), MabTrap kit, HiTrap Protein G HP (GE Healthcare).

All the samples resulting from these different purification techniques were then analysed using classical Western Blot techniques as described for the cell lysates under reducing or non-reducing conditions.

### 3. Design and Methods for Mutating the First Acceptor Splice Site Sequence:

The sequence of the first acceptor splice site was mutated in order to diminish its strength in the same way as it was done for the second acceptor site on the pV1 vector.

Different pairs of primers (sense and antisense) were designed to create different mutants for this site using the Quikchange method (Stratagene). The sequence we chose for these mutations were the sequences of the 8 mutants described above for the pV1 vector and mentioned as MG-72 to MG-15.

The Quikchange reaction was performed on the p1GN-NV vector using the 8 different pairs of primers according to the manufacturer's instructions. The resulting products were used to transform supercompetent TOP10 E. Coli bacteria. Several clones were picked up from LB/agar plates and DNA sequences were determined by sequence analysis in order to identify the desired mutants.

### 4. RT-PCR Analysis on Transfected CHO Cells:

RT-PCR analyses were performed on transfected CHO total RNA to determine the relative amount of each alternatively spliced mRNA. The protocol was the same as described above. The primers used for the PCR amplification of the cDNA fragments were:

Primer 1: (forward primer hybridizing upstream the donor splice site from position 12 to position 31) GAAGTTGGTCGTGAGGCACT (SEQ ID No 71).
Primer 2: (reverse primer hybridizing in the heavy chain sequence between 303 and 324 bases after the start codon) GCAGGTACAGGATGTTCCTGGC (SEQ ID No 74).

At various PCR cycles aliquots of PCR products were loaded on a 2% agarose gel. For each sample, a control PCR reaction was performed using human β-actin primers.

### Results:

### A) Alternative Splicing Using Consensus Sequence for Splice Sites

The first transfection experiment was performed with CHO cells using the p1GN-NV vector containing the consensus sequences for the different splice sites. In a preliminary Western Blot analysis done on the cell lysates only the free light chain was detectable, in a high quantity and no heavy chain. This indicates that, contrary to what was observed with the pV1 vector containing the luciferases, with the p1GN-NV, the expression of the first cistron is much more important than the expression of the second cistron. In this construct the first acceptor splice site seemed to be much more frequently used than the second one by the splicing machinery. That is why we chose to mutate the first acceptor site in order to try and modulate the expression ratio between the light and heavy chains.

N.B.: This result also indicates that alternative splicing depends of the intrinsic sequences of the cistrons cloned in the expression cassette.

### B) RT-PCR Analysis, Identification and Mutation of Cryptic Splice Sites:

The RT-PCR analysis was performed as previously described after RNA extraction from the CHO cells transfected with the p1GN-NV. This experiment was done mostly to confirm that the mRNA transcript resulting from splicing on the first acceptor site was in a large majority compared to the transcript spliced on the second acceptor site. The agarose gels corresponding to the PCR products taken after 30 cycles are shown on FIG. 7a.

The theoretic sizes of the corresponding PCR fragments are:
unspliced transcript: 1350 bp
transcript spliced on the first AS: 1220 bp (light chain)
transcript spliced on the second AS: 370 bp (heavy chain)

The profile of the agarose gel was quite different from what was expected. Indeed, many bands were observed. One major band seemed to correspond to the transcript spliced on the first AS and no band corresponding to the transcript spliced on the second AS, thus confirming the Western blot results. However, several other bands from different intermediate sizes were detected, most of them quite intense. This tends to indicate that many aberrant splicing events frequently occurred on the pre-mRNA transcribed from the p1GN-NV, generating a pool of mis-spliced transcripts that lead to the expression of truncated polypeptides. Because these aberrant splicing events seemed to be very frequent, we had to find a way to reduce them as much as possible in order to maximize the proportion of correctly spliced transcript, and thus improve the expression yield of the proteins of interest. This was done by identifying the cryptic splice sites implicated in this aberrant splicing and by mutating them. The procedure was the following:

The different bands visualized on the agarose gel showed in FIG. 7a were cut and DNA fragments were purified using the Nucleospin extract kit (Macherey-Nagel). Each purified fragment was cloned in the TOPO-TA vector and the resulting vectors were submitted to sequence analysis. The sequences of the different fragments were then aligned on the theoretic sequence of the p1GN-NV vector in order to localize precisely the positions were the corresponding mRNA transcript was cut, thus indicating the positions of the cryptic splice sites involved in aberrant splicing. This procedure was repeated for all different species of spliced transcripts. It allowed us to identify several cryptic splice sites, donor and acceptor sites, in the coding sequence of the light chain but also in the non-coding sequence, i.e. in the 5′UTR or in the intercistronic region. A cryptic acceptor site may splice with the constitutive donor site, a cryptic donor site may splice with the second constitutive acceptor site, or two cryptic splice sites may splice together as shown in FIG. 8 (N.B.: splice sites referred to as “constitutive” splice sites are the sites described in the construction of the vector). The relative intensity of each band visualized on agarose for each fragment gives an indication of the frequency of each aberrant splicing event. As shown in FIG. 7a, some of them are very frequent, and some others happen more rarely.

However, every mis-spliced mRNA leads to a truncated protein and must therefore be avoided. As explained above, several cryptic splice sites were then identified after the first RT-PCR experiment. One of them in particular, a donor site located in the ten last bases just before the STOP codon of the light chain, was found to splice with the second constitutive acceptor site on almost 90% of the spliced transcripts. We mutated this site first in order to suppress this major aberrant splicing. This was done using the Quikchange method (Stratagene) and a pair of complementary primers designed to modify the sequence of the cryptic site without changing the amino-acid sequence of the translated polypeptide.

Initial sequence:

(SEQ ID N^{o} 75)

G AGC TTC AAC AGG AAT GAG TGT TAG TCTAGATTCTTGTCG.
Sense primer:

(SEQ ID N^{o} 76)

G AGC TTC AAC CGC AAT GAA TGC TAA TCTAGATTCTTGTCG.

The resulting mutated vector, called p1GN-NV-ml was used to transiently transfect CHO cells and the RT-PCR analysis (including splice sites identification) was performed on total RNA extracted from these cells as described before. As said before, splice sites may be activated if a mutation alters or removes a genuine nearby site. Consequently, each time a cryptic site is mutated, new cryptic splice sites, not activated in the previous configuration, might appear; cryptic sites that seemed to be rarely used in the first experiment, may become major splice sites after mutation of a nearby cryptic site. That is why we had to make a new RT-PCR experiment after each mutation. The results of the second RT-PCR are shown in FIG. 7b. The profile is quite different from the previous one: one band corresponding to the mRNA spliced on the first AS, one band for the mRNA spliced on the second AS and fewer extra bands indicating that aberrant splicing was considerably lowered. Sequence analysis revealed many cryptic sites, most of them had already been identified in the first experiment, but the frequency of use was changed. We mutated the site that appeared to be the major one as indicated above with appropriate primers. The whole experience was repeated identically many times. Several major sites and minor sites were mutated (when the two bases consensus of a splice site could not be mutated without changing amino-acid sequence, we tried to modify other bases in the site environment in order to weaken the site to a maximum) until the RT-PCR profile was as “clean” as expected, i.e. only two bands corresponding to constitutive alternatively spliced mRNAs. As mutations were performed, fewer sites appeared, they were less used and finally aberrant splicing seemed to become quite rare. An example of the final RT-PCR profile obtained is shown in FIG. 7c. The two major bands observed correspond to the constitutively spliced mRNAs and no extra bands are detected.

The 1220-bp band, corresponding to the mRNA spliced on the first AS is much more intense than the 370 bp band, corresponding to splicing on the second AS. This observation according to the preliminary Western Blot results confirmed that the first acceptor site is much more used than the second one, and that this site needed to be mutated in order to modulate the expression ratio between light and heavy chains.

N.B.: The luciferases genes used for the construction of the pV1 vector had been previously mutated in our laboratory to suppress all the putative cryptic splice sites. The RT-PCR experiments confirmed that no other cryptic splice site was recognized by the splicing machinery.

Mutation of the cryptic splice sites appeared to be an indispensable step, that has to be done carefully for each new gene to be expressed from the vector of the invention, in order to optimize the production yield.

### C) Modulation of Alternative Splicing by Splice Sites Engineering

In order to regulate the ratio between the two different mRNAs (and consequently regulate the relative expression of the light and heavy chains) we mutated the sequence of the first acceptor splice site, as described previously, and tested if these modifications had an impact on the choice of the acceptor site selected by the splicing machinery. The sequences chosen were the sequences of the 8 mutants described above for the pV1 vector and mentioned as MG-72 to MG-15.

### a) Western Blot Analysis

These mutants, when used to transfect cells, generated important variations in the relative expression of the two chains, according to what was detected both in the cell lysates and in the cell culture supernatants. An example of these variations is shown in FIG. 9. Two particular mutants of the first acceptor splice site (J1 and H2) are compared on this figure to the construction harbouring the consensus sequence for the first acceptor site (K3). The samples were not submitted to reduction, thus allowing observation of the whole antibody molecule (150 kDa), assembly intermediates (125, 100, 75 kDa) and unassembled free chains (50 and 25 kDa). For the K3 vector, free light chain is detected, no free heavy chain, and small amounts of assembly intermediates and whole antibody. Mutant J1, compared to K3, shows similar quantities of free light and heavy chain and larger amounts of assembly intermediates and whole antibody. On the opposite, mutant H2 shows a strong surexpression of heavy chain, no light chain, no whole antibody and high amounts of heavy chain multimers (100 kDa).

These observations can be interpreted this way:
for construction K3: high expression of the light chain and very weak expression of the heavy chain. Titration of the small quantities of heavy chain by the light chain in excess to form whole antibody (confirmed by the analysis on culture supernatant: high amounts of free light chain secreted, very small amounts of whole antibody secreted).
for mutant H2: high expression of heavy chain and no expression of the light chain. Multimerization of heavy chain in excess that cannot be secreted (nothing detected in supernatant).
for mutant J1: balanced expression of the two chains, which assemble into whole antibody (whole antibody also detected in the supernatant).

These results indicate that mutation of the first acceptor splice site allows modulation of the ratio between the two chains. Whole antibody can be expressed and secreted with different efficiencies; antigen binding properties can then be determined using ELISA or Biacore experiments for example. For each antibody to be expressed with the vector of the invention, an appropriate mutant has to be identified among the mutants library constructed, i.e. the mutant that gives the higher amounts of secreted, correctly folded and functional antibody molecule.

### b) RT-PCR Analysis

A RT-PCR analysis was performed on the RNA from cells transfected with the different mutants of the first constitutive acceptor splice site. This analysis revealed, as predictable, that decreasing the strength of the first constitutive acceptor site resulted in the activation of several cryptic splice sites. Thus, a few splice sites that were not found on the first experiments or that were in minority were identified and then mutated with the same protocol as described above.

### REFERENCES

Throughout this application, various references describe the state of the art to which this invention pertains. The disclosures of these references are hereby incorporated by reference into the present disclosure.
Adamson T E, Price D H, Cotranscriptional processing of drosophila histone mRNAs. Mol Cell Biol. 2003, 23: 4046-4055.
Creancier L, Morello D, Mercier P, Prats A C, Fibroblast growth factor 2 internal ribosomal entry site (IRES) activity ex vivo and in transgenic mice reveals a stringent tissue-specific regulation. J Cell Biol. 2000, 150: 275-281.
Edwalds-Gilbert G, Veraldi K L, Milcarek C, Alternative poly(A) site selection in complex transcription units: means to an end? Nucleic Acids Res 1997, 25: 2547-2561.
Hall-Pogar T, Zhang H, Thian B, Lutz C S, Alternative polyadenylation of cyclooxygenase 2. Nucleic Acids Res. 2005, 33: 2565-2579.
Hellen C U, Sarnow P, Internal ribosome entry sites in eukaryotic mRNA molecules. Genes Dev. 2001 Jul. 1; 15(13):1593-612.
Moreira A, Wollerton M, Monks J, Proudfoot N J, Upstream sequence elements enhance poly(A) site efficiency of the C2 complement gene and are phylogenetically conserved. EMBO J. 1995, 14: 3809-3819.
Niwa M, MacDonald C C, Berget S M, Are vertebrate exons scanned during splice-site selection? Nature 1992, 360: 277-280.
Osheim Y N, Proudfoot N J, Beyer A L, EM visualization of transcription by RNA polymerase II: downstream termination requires a poly(A) signal but not transcript cleavage. Mol Cell 1999, 3: 379-387.
Proudfoot N J, How RNA polymerase II terminates transcription in higher eucaryotes. Trends Biochem. Sci. 1989, 14: 105-110.
Proudfoot N J, Ending the message is not simple. Cell 1996, 87: 779-781.
Proudfoot N J, Furger A, Dye M J, Integrating mRNA processing with transcription. Cell 2002, 108: 501-512.
West S, Gromak N, Proudfoot N J, Human 5′-3′ exonuclease Xrn2 promotes transcription termination at co-transcriptional cleavage sites. Nature 2004, 432: 522-525.

1. An expression cassette comprising in 5′ to 3′ downstream direction: a promoter; a sequence transcribed in a 5′ untranslated region (5′UTR); a donor splice site; an intron; a first acceptor splice site; a first cistron encoding a first polypeptide; a second acceptor splice site; a second cistron encoding a second polypeptide; an internal ribosome entry site (IRES) operably linked to a selection marker; and a sequence transcribed in a 3′ untranslated region (3′UTR) including a polyadenylation signal,
wherein the polyadenylation signal is unique, wherein the promoter is operably linked to the first and second cistron and wherein upon entry into an eukaryotic host cell, said donor splice site splices with said first acceptor splice site, forming a spliced transcript which enables transcription of said first cistron encoding said first polypeptide, and said second acceptor splice site forming a spliced transcript which permits transcription of said second cistron encoding said second polypeptide.

2. The expression cassette of claim 1, wherein said expression cassette further comprises between said second cistron and said IRES one or more additional acceptor splice sites operably linked to an additional cistron encoding an additional polypeptide wherein upon entry into an eukaryotic host cell, said donor splice site splices with said additional splice acceptor, forming an additional spliced transcript which enables transcription of said additional cistron encoding said additional polypeptide.

3. The expression cassette according to claim 1, wherein at least one of said acceptor splice sites comprises any one of the sequences selected from the group consisting of SEQ ID NOS: 1-64.

4. The expression cassette according to claim 1, wherein said polypeptides encoded by said cistrons form a multimeric protein.

5. The expression cassette according to claim 1, wherein said first polypeptide is an antibody heavy chain or a fragment thereof and said second polypeptide is an antibody light chain or a fragment thereof.

6. The expression cassette according to claim 1, wherein said first polypeptide is an antibody light chain or a fragment thereof and said second polypeptide is an antibody heavy chain or a fragment thereof.

7. The expression cassette according to claim 1, wherein said cistrons are replaced by other cistrons in the expression cassette using restriction sites located on both sides of said cistrons.

8. A polynucleotide comprising an expression cassette according to claim 1.

9. A viral vector comprising the polynucleotide of claim 8.

10. A polynucleotide comprising an expression cassette according to claim 7.

11. A eukaryotic host cell containing a polynucleotide according to claim 8.

12. The eukaryotic host cell of claim 11, wherein the polynucleotide is integrated into the chromosomal DNA of said eukaryotic host cell.

13. The cell of claim 11, wherein said eukaryotic host cell is selected form the group consisting of a mammalian cell, an insect cell and a yeast cell.

14. A method of producing polypeptides, the method comprising culturing a eukaryotic host cell according to claim 11 in a culture and isolating said polypeptides encoded by said cistrons from the culture.

15. A polynucleotide or a viral vector according to claim 8 for use in a method for treatment of the human or animal body by therapy wherein said cistrons encode therapeutic polypeptides or encode for polypeptides which form a therapeutic heteromultimeric protein.

16. Method of treating a patient in need thereof by gene therapy, which comprises administering to the patient an effective amount of a drug comprising a polynucleotide or a viral vector according to claim 15.


SEQUENCE LISTING (77 sequences)

SEQ ID NO: 1 - DNA, 17 bp, artificial
  acceptor splice site sequence
cctttctctctataggt

SEQ ID NO: 2 - DNA, 17 bp, artificial
  acceptor splice site sequence
cctttctctctaaaggt

SEQ ID NO: 3 - DNA, 17 bp, artificial
  acceptor splice site sequence
cctttctctctagaggt

SEQ ID NO: 4 - DNA, 17 bp, artificial
  acceptor splice site sequence
cctttctctctacaggt

SEQ ID NO: 5 - DNA, 17 bp, artificial
  acceptor splice site sequence
cctttctctccacaggt

SEQ ID NO: 6 - DNA, 17 bp, artificial
  acceptor splice site sequence
cctttctctccataggt

SEQ ID NO: 7 - DNA, 17 bp, artificial
  acceptor splice site sequence
cctttctctccaaaggt

SEQ ID NO: 8 - DNA, 17 bp, artificial
  acceptor splice site sequence
cctttctctccagaggt

SEQ ID NO: 9 - DNA, 17 bp, artificial
  acceptor splice site sequence
cctttctctcgagaggt

SEQ ID NO: 10 - DNA, 17 bp, artificial
  acceptor splice site sequence
cctttctctcgacaggt

SEQ ID NO: 11 - DNA, 17 bp, artificial
  acceptor splice site sequence
cctttctctcgaaaggt

SEQ ID NO: 12 - DNA, 17 bp, artificial
  acceptor splice site sequence
cctttctctcgataggt

SEQ ID NO: 13 - DNA, 17 bp, artificial
  acceptor splice site sequence
cctttctctcaaaaggt

SEQ ID NO: 14 - DNA, 17 bp, artificial
  acceptor splice site sequence
cctttctctcaacaggt

SEQ ID NO: 15 - DNA, 17 bp, artificial
  acceptor splice site sequence
cctttctctcaataggt

SEQ ID NO: 16 - DNA, 17 bp, artificial
  acceptor splice site sequence
cctttctctcaagaggt

SEQ ID NO: 17 - DNA, 17 bp, artificial
  acceptor splice site sequence
ccttcctctctataggt

SEQ ID NO: 18 - DNA, 17 bp, artificial
  acceptor splice site sequence
ccttcctctctaaaggt

SEQ ID NO: 19 - DNA, 17 bp, artificial
  acceptor splice site sequence
ccttcctctctagaggt

SEQ ID NO: 20 - DNA, 17 bp, artificial
  acceptor splice site sequence
ccttcctctctacaggt

SEQ ID NO: 21 - DNA, 17 bp, artificial
  acceptor splice site sequence
ccttcctctccacaggt

SEQ ID NO: 22 - DNA, 17 bp, artificial
  acceptor splice site sequence
ccttcctctccataggt

SEQ ID NO: 23 - DNA, 17 bp, artificial
  acceptor splice site sequence
ccttcctctccaaaggt

SEQ ID NO: 24 - DNA, 17 bp, artificial
  acceptor splice site sequence
ccttcctctccagaggt

SEQ ID NO: 25 - DNA, 17 bp, artificial
  acceptor splice site sequence
ccttcctctcgagaggt

SEQ ID NO: 26 - DNA, 17 bp, artificial
  acceptor splice site sequence
ccttcctctcgacaggt

SEQ ID NO: 27 - DNA, 17 bp, artificial
  acceptor splice site sequence
ccttcctctcgaaaggt

SEQ ID NO: 28 - DNA, 17 bp, artificial
  acceptor splice site sequence
ccttcctctcgataggt

SEQ ID NO: 29 - DNA, 17 bp, artificial
  acceptor splice site sequence
ccttcctctcaaaaggt

SEQ ID NO: 30 - DNA, 17 bp, artificial
  acceptor splice site sequence
ccttcctctcaacaggt

SEQ ID NO: 31 - DNA, 17 bp, artificial
  acceptor splice site sequence
ccttcctctcaataggt

SEQ ID NO: 32 - DNA, 17 bp, artificial
  acceptor splice site sequence
ccttcctctcaagaggt

SEQ ID NO: 33 - DNA, 17 bp, artificial
  acceptor splice site sequence
ccttactctctataggt

SEQ ID NO: 34 - DNA, 17 bp, artificial
  acceptor splice site sequence
ccttactctctaaaggt

SEQ ID NO: 35 - DNA, 17 bp, artificial
  acceptor splice site sequence
ccttactctctagaggt

SEQ ID NO: 36 - DNA, 17 bp, artificial
  acceptor splice site sequence
ccttactctctacaggt

SEQ ID NO: 37 - DNA, 17 bp, artificial
  acceptor splice site sequence
ccttactctccacaggt

SEQ ID NO: 38 - DNA, 17 bp, artificial
  acceptor splice site sequence
ccttactctccataggt

SEQ ID NO: 39 - DNA, 17 bp, artificial
  acceptor splice site sequence
ccttactctccaaaggt

SEQ ID NO: 40 - DNA, 17 bp, artificial
  acceptor splice site sequence
ccttactctccagaggt

SEQ ID NO: 41 - DNA, 17 bp, artificial
  acceptor splice site sequence
ccttactctcgagaggt

SEQ ID NO: 42 - DNA, 17 bp, artificial
  acceptor splice site sequence
ccttactctcgacaggt

SEQ ID NO: 43 - DNA, 17 bp, artificial
  acceptor splice site sequence
ccttactctcgaaaggt

SEQ ID NO: 44 - DNA, 17 bp, artificial
  acceptor splice site sequence
ccttactctcgataggt

SEQ ID NO: 45 - DNA, 17 bp, artificial
  acceptor splice site sequence
ccttactctcaaaaggt

SEQ ID NO: 46 - DNA, 17 bp, artificial
  acceptor splice site sequence
ccttactctcaacaggt

SEQ ID NO: 47 - DNA, 17 bp, artificial
  acceptor splice site sequence
ccttactctcaataggt

SEQ ID NO: 48 - DNA, 17 bp, artificial
  acceptor splice site sequence
ccttactctcaagaggt

SEQ ID NO: 49 - DNA, 17 bp, artificial
  acceptor splice site sequence
ccttgctctctataggt

SEQ ID NO: 50 - DNA, 17 bp, artificial
  acceptor splice site sequence
ccttgctctctaaaggt

SEQ ID NO: 51 - DNA, 17 bp, artificial
  acceptor splice site sequence
ccttgctctctagaggt

SEQ ID NO: 52 - DNA, 17 bp, artificial
  acceptor splice site sequence
ccttgctctctacaggt

SEQ ID NO: 53 - DNA, 17 bp, artificial
  acceptor splice site sequence
ccttgctctccacaggt

SEQ ID NO: 54 - DNA, 17 bp, artificial
  acceptor splice site sequence
ccttgctctccataggt

SEQ ID NO: 55 - DNA, 17 bp, artificial
  acceptor splice site sequence
ccttgctctccaaaggt

SEQ ID NO: 56 - DNA, 17 bp, artificial
  acceptor splice site sequence
ccttgctctccagaggt

SEQ ID NO: 57 - DNA, 17 bp, artificial
  acceptor splice site sequence
ccttgctctcgagaggt

SEQ ID NO: 58 - DNA, 17 bp, artificial
  acceptor splice site sequence
ccttgctctcgacaggt

SEQ ID NO: 59 - DNA, 17 bp, artificial
  acceptor splice site sequence
ccttgctctcgaaaggt

SEQ ID NO: 60 - DNA, 17 bp, artificial
  acceptor splice site sequence
ccttgctctcgataggt

SEQ ID NO: 61 - DNA, 17 bp, artificial
  acceptor splice site sequence
ccttgctctcaaaaggt

SEQ ID NO: 62 - DNA, 17 bp, artificial
  acceptor splice site sequence
ccttgctctcaacaggt

SEQ ID NO: 63 - DNA, 17 bp, artificial
  acceptor splice site sequence
ccttgctctcaataggt

SEQ ID NO: 64 - DNA, 17 bp, artificial
  acceptor splice site sequence
ccttgctctcaagaggt

SEQ ID NO: 65 - DNA, 44 bp, artificial
  primer
aaacctaggatccatgtacccatacgatgttccagattacgctn

SEQ ID NO: 66 - DNA, 57 bp, artificial
  primer
ccttaattaacacctgtggagagaaaggaaaagtggatgtcagtaagaccgcggccg

SEQ ID NO: 67 - DNA, 47 bp, artificial
  primer
ccttaattaagctagcatgtacccatacgatgttccagattacgctn

SEQ ID NO: 68 - DNA, 21 bp, artificial
  primer
gaagatctcccggggatatcn

SEQ ID NO: 69 - DNA, 50 bp, artificial
  primer
ggccgcggtcttactgacatccacttttccttnctcttnanaggtgtaat

SEQ ID NO: 70 - DNA, 45 bp, artificial
  primer
taacacctntnaagagnaaggaaaagtggatgtcagtaagaccgc

SEQ ID NO: 71 - DNA, 20 bp, artificial
  primer
gaagttggtcgtgaggcact

SEQ ID NO: 72 - DNA, 21 bp, artificial
  primer
cataaataagaagaggccgcg

SEQ ID NO: 73 - DNA, 20 bp, artificial
  primer
gcaattgttccaggaaccag

SEQ ID NO: 74 - DNA, 137 bp, artificial
  intron sequence
caggtaagtatcaaggttacaagacaggtttaaggagaccaatagaaactgggcttgtcgagacagagaagactcttgcgtttctgataggcacctattggtcttactgacatccactttgcctttctctccacagg

SEQ ID NO: 75 - DNA, 40 bp, Artificial
  primer
gagcttcaacaggaatgagtgttagtctagattcttgtcg

SEQ ID NO: 76 - DNA, 40 bp, Artificial
  primer
gagcttcaaccgcaatgaatgctaatctagattcttgtcg

SEQ ID NO: 77 - DNA, 137 bp, artificial; identical to SEQ ID NO: 74
  intron